(19) United States
(12) Patent Application Publication
(10) Pub. No.: US 2007/0292536 A1
(43) Pub. Date: Dec. 20, 2007

(54) COMPOSITION AND METHOD FOR TREATING PATIENTS WITH HIGH NEUROTRANSMITTER LEVELS

(76) Inventor: Gottfried Kellermann, Osceola, WI (US)

Correspondence Address: RONALD L. HOFER, 122 LINDBERGH LANE, MOORESVILLE, NC, 28117, US

(21) Appl. No.: 11/424,703

(22) Filed: Jun. 16, 2006

(51) Int. Cl. A61K 36/185; A61K 31/685; A61K 31/185; A61K 31/198

(52) U.S. Cl. 424/725; 514/553; 514/78; 514/561

(57) ABSTRACT

A patient having high neurotransmitter levels is treated by administering an effective amount of a treatment composition comprising Rhodiola Rosea and phosphatidylserine in a pharmaceutically acceptable carrier.

### BACKGROUND OF THE INVENTION

The present invention relates to a composition and method for treating patients with high neurotransmitter levels. More specifically, the present invention relates to a composition and method for treating patients with symptoms attributable to high neurotransmitter levels, which composition and method are effective to reduce neurotransmitter levels and reduce or eliminate symptoms caused by high neurotransmitter levels.

In recent years there has been considerable research relating to neurotransmitters, the functions of neurotransmitters, methods of manipulation of neurotransmitters, and the effect of manipulation of neurotransmitters on various disorders. Neurotransmitters and many of the compounds used in manipulation of neurotransmitter levels are naturally occurring materials which have been studied for many years. However, much of the science relating to neurotransmitters remains to be discovered. Some patients exhibit symptoms which are believed to be related to neurotransmitter disorders but which are not effectively treated by known methods of manipulating neurotransmitters. Other patients are treated with neurotransmitter manipulating compositions only to have new neurological symptoms appear.

For example, I have found that patients who exhibit symptoms such as ADHD, aggressiveness, stress/anxiety, etc, often have elevated levels of the excitatory neuro-transmitters, for example, epinephrine, norepinephrine, dopamine, etc. In many of these patients dosing the patients with amino acids that increase or enhance inhibitory neurotransmitters will reduce the levels of excitatory neurotransmitters as evidenced by declines in the urinary level of these neurotransmitters and reduce the severity of the patient's symptoms. There are however, some patients that do not have positive outcomes with this regimen and therefore require additional or alternative therapies to control their excitatory clinical symptoms as well as additional or alternative therapies to control their neurotransmitter levels.

In accordance with the present invention, a composition and method for treatment of patients with high neurotransmitter levels is provided. The composition and method also provide an alternative treatment for those patients who do not respond to conventional treatment with amino acids to reduce their levels of excitatory neurotransmitter levels. The composition and method may also be used to offset the side effect of some medications which raise neurotransmitter levels or to compensate for over-excitation resulting from use of medications. Further understanding of the present invention will be had from the following specification taken in conjunction with the claims appended hereto.

### SUMMARY OF THE INVENTION

In accordance with the method of the present invention, a patient having high neurotransmitter levels is treated by administering an effective amount of a treatment composition to the patient. A treatment composition of the present invention comprises an effective amount of Rhodiola Rosea and an effective amount of phosphatidylserine in a pharmaceutically acceptable carrier.

### DETAILED DESCRIPTION OF THE INVENTION

Examples of symptoms of over-stimulation in a patient include symptoms such as abnormal aggressiveness, excessive stress or anxiety, recalcitrant behavior, restlessness, or agitation. Over-stimulated patients might be diagnosed with certain disorders such as ADHD (attention deficit hyperactivity disorder), obsessive/compulsive disorder, oppositional/defiance disorder, or internalizing or externalizing behavior disorders. Patients who exhibit symptoms of over-stimulation often have elevated levels of excitatory neurotransmitters such as epinephrine, norepinephrine, dopamine, or other excitatory neurotransmitters.

In many of these patients dosing the patients with amino acids that increase or enhance inhibitory neurotransmitters will reduce their levels of excitatory neuro-transmitters, as evidenced by declines in the urinary level of these neurotransmitters, and reduce the severity of the patient's symptoms. There are however, some patients that do not have positive outcomes with this regimen and therefore require additional or alternative therapies to control their excitatory clinical symptoms as well as additional or alternative therapies to control their neurotransmitter levels. The method of the present invention is particularly useful for these patients. A treatment that will reduce overt overstimulation as well as poor psychological self-control is needed for treatment resistant patients. Also, an initial therapy using amino acids or neurotransmitter precursors to affect neurotransmitter levels may also include other medications which have an adverse side effect of over-stimulation. The present invention may also be used to treat over-stimulation that results as a side effect of another treatment.

In accordance with the present invention, a composition and treatment method is provided for patients with high neurotransmitter levels. The composition and method may be used as an alternative treatment composition and method for those patients who do not respond to conventional treatment with amino acids to reduce the levels of excitatory neurotransmitter levels as well as a composition and method for patients who experience an adverse side effect of over-stimulation or a conventional medical treatment used to treat symptoms of understimulation. Conventional amino acid treatment involves administering inhibitory amino acids and/or precursors to inhibitory neurotransmitters, for example, 5-HTP, taurine, or theanine, alone or in combination with other neurotranmsmitter precursors, for example, tyrosine, phenylalanine, histamine, L-dopa (Mucuna pruriens). Conventional medical treatments include but are not limited to the administration of stimulant medications such as methylphenidate, Adderall, dextroamphetamine, etc., or neurotransmitter reuptake inhibitors such as atomoxepine, escitalopram, venlefaxine, etc. or anxiolytics such as benzodiazepines, buspirone, beta-blockers, alpha-agonists, or the administration of other psychotropic medications.

It has been found that patients who do not respond to conventional treatment can be successfully treated to reduce their neurotransmitter levels and the severity of their symptoms by administrating to the patients a treatment composition which comprises an effective amount of Rhodiola Rosea and an effective amount of phoshatidylserine.

Rhodiola Rosea is found naturally in the northern regions of Europe, Scandinavia, Lapland and Alaska. Rhodiola Rosea is a natural plant substance which has long been known as a potent adaptogen, i.e., a natural plant substance which increases the body's non specific resistance, normalizes the functions of the body, and has no adverse side effects. Rhodiola Rosea has been categorized as an adaptogen by Russian researchers due to its observed ability to increase resistance to a variety of chemical, biological, and physical stressors. Its claimed benefits include antidepressant, anticancer, cardioprotective, and central nervous system enhancement (stimulation). Rhodiola Rosea is commercially available from KDN-Vita International Inc. of Hillsborough, NJ.

Phosphatidylserine is a phospholipid, 1,2-diacyl-sn-glycerol-(3)-L-phosphoserine. Phosphatidylserine is a phospholipid nutrient, sometimes referred to a “PS”. PS is essential to the functioning of all the cells of the body, but is most concentrated in the brain. Its relative abundance in this organ reflects its proven involvement in an assortment of nerve cell functions, including nerve transmitter release and synaptic activity. Clinical studies have suggested that PS can support brain functions that tend to decline with age. In the present invention, phosphatidylserine is included to alter the regulatory mechanisms affecting neurotransmitter release and activation. Other components with similar abilities to effect excitatory neurotransmitter release via antagonist or agonist properties may be substituted for phosphatidylserine. This may include phosphatidylserine analogs like phosphorylated serine and well as synthetic or naturally occurring peptides that affect the release of excitatory neurotransmitters. Phosphatidylserine is commercially available from Chemi Nutra, White Bear Lake, Minn.

It is contemplated that a treatment composition of the present invention will comprise an effective amount of each active ingredient, i.e., Rhodiola Rosea and phosphatidylserine. The active ingredients may be in any pharmaceutically acceptable carrier and the composition can by made by any suitable method. The exact nature of the composition will depend upon its method of administration to the patient. For example, a composition intended to be administered orally can be made by simply mixing the active ingredients and encapsulating them.

Suitable concentrations of each active ingredient in the composition can vary. In a preferred embodiment of this invention, a daily dose of about 600 mg of Rhodiola rosea and about 400 mg of phosphatidyl serine are administered to a patient. Rhodiola rosea is a natural product comprising active components Rosavin, rosarin, rosin, and salidrosides. The 600 mg of Rhodiola rosea extract used in the example set forth below contained about 64.8 mg Rosavin, 20.4 mg of Rosarin, 13.8 mg Rosin, and 31.2 mg Salidrosides. Daily doses of active components which have been found to be effective are: for Rosavin, rosarin, rosin, salidrosides are 29.4-64.8 mg Rosavin, 10.0-20.4 mg of Rosarin, 5.92-13.8 mg Rosin, and 10.9-31.2 mg Salidrosides in a total of 400-600 mg of Rhodiola rosea extract. The weight ratio of Rhodiola rosea to phosphatidylserine can vary over a wide range. Weight ratios of Rhodiola rosea to phosphatidylserine of from 1:1 to 3:1 are examples of ratios which are believed to be suitable for use in the present invention.

The treatment composition may be administered to the patient by any suitable means including oral administration via pills, capsules, liquid suspensions and liposomal formulas. Alternatively, the composition may be administered to the patient transdermally via topically applied (transmucosally, suppositories, etc.) creams, lotions, gels etc. containing effective permeation enhancement agents.

By the term “effective amount” is meant an amount which is sufficient to have the desired therapeutic effect, i.e., a reduction in the severity of the symptoms. I have found that oral doses of Rhodiola Rosea (600 mg) once per day and phosphatidylserine (200 mg twice per day) are effective in patients that have not responded to initial treatment with inhibitory amino acids such as 5-HTP, taurine, or theanine. High doses of Rhodiola constituents are required to obtain the inhibitory effect of this invention. Low to medium doses of Rhodiola would have an unwanted stimulation effect. An insufficient dose of Rhodiola rosea would be excitatory and indicate that the dose is ineffective. Commonly used doses of Rhodiola are too low to be inhibitory due to the low concentrations of active constituents combined with low dosing. Sufficient doses to be inhibitory require that large doses of products with low levels of active components or normal doses of products with high levels of the active constituents be used.

The treatment method of the present invention is useful to treat or ameliorate various symptoms in patients having high neurotransmitter levels. The method is well suited for patients for whom conventional treatment has failed to obtain a sufficient lowering of neurotransmitter levels. Thus, it is contemplated that a patient will first be diagnosed by a medical professional as having symptoms related to high neurotransmitter levels. High neurotransmitter levels may occur due to either an increase or decrease in the activity of a particular neurotransmitter, or due to an under- or over-regulation due to impaired feed back mechanisms. Due to the interactive nature of neurotransmitters, excess stimulation may occur due to either high activity of one or more excitatory neurotransmitter or a lack of inhibitory neurotransmitters.

It is further contemplated that in some cases patients will be selected by discovering that conventional treatments containing inhibitory amino acids such as 5-HTP, taurine, or theanine, has unsatisfactory results. The selected patient will then be treated in accordance with the method of the present invention. After such treatment, laboratory tests for this patent will evidence a reduction of the excitatory neurotransmitters and dramatic reductions in clinical symptoms.

Treatment compositions can include other optional ingredients, both active and inactive, such as taurine, Glycine, agmatine, phosphorlated serine, tyrosine, 1-dopa, Mucuna pruriens, phenylalanine, stimulants, anxiolytics).

Further understanding of this invention may be had from the following specific example.

### EXAMPLE

A (9 year old male) patient has symptoms of high anxiety and poor concentration and is tested and found to have the following high neurotransmitter levels:

| | | | | | After |
|---|---|---|---|---|---|
| Neuro- | | | After | After | treatment |
| transmitter | Observed | Base- | Primary | Secondary | with |
| Ug/grCr | Ranges | line | treatment | treatment | invention |
| Epinephrine | 1.5–21.0 | 18.4 | — | — | — |
| Norepinephrine | 15.5–106 | 135.2 | — | — | — |
| Serotonin | 41–275 | 81.7 | — | — | — |

### The patient is treated by the following regime (primary treatment) in an attempt to reduce the symptoms and neurotransmitter levels.

### Total Daily Therapy in Capsule Form:

| | | |
|---|---|---|
| 5-Hydroxytryptophan | 130 | mg |
| Taurine | 320 | mg |
| Theanine | 630 | mg |
| N-acetylcysteine | 320 | mg |
| Vitamin B6 (Pyridoxine HCL & P-5′-P) | 17 | mg |
| Folic Acid | 130 | mcg |
| Vitamin C | 27 | mg |
| Magnesium glycinate | 130 | mg |
| Vitamin B12 | 20 | mcg |
| Zinc | 7 | mg |
| Selenomethionine | 27 | mcg |

### The patient is tested and found to have the following high neurotransmitter levels.

| | | | | | After |
|---|---|---|---|---|---|
| Neuro- | | | After | After | treatment |
| transmitter | Observed | Base- | Primary | Secondary | with |
| Ug/grCr | Ranges | line | treatment | treatment | invention |
| Epinephrine | 1.5–21.0 | 18.4 | 12.9 | — | — |
| Norepinephrine | 15.5–106 | 135.2 | 64.9 | — | — |
| Serotonin | 41–275 | 81.7 | 259.2 | — | — |

The patient continues to have the symptoms set forth above and is now treated by the following regime (secondary treatment) in capsule form in an attempt to reduce the symptoms and neurotransmitter levels:

| | | |
|---|---|---|
| 5-Hydroxytryptophan | 130 | mg |
| Taurine | 320 | mg |
| Theanine | 630 | mg |
| N-acetylcysteine | 745 | mg |
| Vitamin B6 (Pyridoxine HCL & P-5′-P) | 30 | mg |
| Folic Acid | 200 | mcg |
| Vitamin C | 127 | mg |
| Magnesium glycinate | 130 | mg |
| Vitamin B12 | 20 | mcg |
| Zinc | 7 | mg |
| Selenomethionine | 64 | mcg |
| Calcium (calcium citrate) | 37 | mg |
| Mucuna pruriens | 100 | mg |
| N-acetyl Tyrosine | 100 | mg |

### After this secondary treatment, the patient continues to have the symptoms set forth above and is again tested for neurotransmitter levels with the following results.

| | | | | | After |
|---|---|---|---|---|---|
| Neuro- | | | After | After | treatment |
| transmitter | Observed | Base- | Primary | Secondary | with |
| Ug/grCr | Ranges | line | treatment | treatment | invention |
| Epinephrine | 1.5–21.0 | 18.4 | 12.9 | 12.5 | — |
| Norepinephrine | 15.5–106 | 135.2 | 64.9 | 84.5 | — |
| Serotonin | 41–275 | 81.7 | 259.2 | 310.0 | — |

The patient continues to have the symptoms set forth above and is treated with the following regime (the invention) in capsule form:

| | | |
|---|---|---|
| 5-Hydroxytryptophan | 130 | mg |
| Taurine | 320 | mg |
| Theanine | 630 | mg |
| N-acetylcysteine | 745 | mg |
| Vitamin B6 (Pyridoxine HCL & P-5′-P) | 30 | mg |
| Folic Acid | 200 | mcg |
| Vitamin C | 127 | mg |
| Magnesium glycinate | 130 | mg |
| Vitamin B12 | 20 | mcg |
| Zinc | 7 | mg |
| Selenomethionine | 64 | mcg |
| Calcium (calcium citrate) | 37 | mg |
| Mucuna pruriens | 100 | mg |
| N-acetyl Tyrosine | 100 | mg |
| Phosphotidylserine | 200 | mg |
| Rhodiola rosea | 400 | mg |

The patient shows a significant reduction in the severity of the above symptoms and is tested for neurotransmitter levels with the following results:

| | | | | | After |
|---|---|---|---|---|---|
| Neuro- | | | After | After | treatment |
| transmitter | Observed | Base- | Primary | Secondary | with |
| Ug/grCr | Ranges | line | treatment | treatment | invention |
| Epinephrine | 1.5–21.0 | 18.4 | 12.9 | 12.5 | 6.1 |
| Norepinephrine | 15.5–106 | 135.2 | 64.9 | 84.5 | 30.9 |
| Serotonin | 41–275 | 81.7 | 259.2 | 310.0 | 342.5 |

The above results show a significant reduction in neurotransmitter levels.

What is claimed is:

1. A method of treating a patient with high neurotransmitter levels, the method comprising administering an effective amount of Rhodiola Rosea and an effective amount of phosphatidylserine to the patient.

2. The method of claim 1, wherein said Rhodiola Rosea and said phosphatidylserine are administered in the same composition.

3. A treatment composition comprising an effective amount of Rhodiola Rosea and an effective amount of phosphatidylserine.

4. The treatment composition of claim 3, wherein said Rhodiola Rosea and said phosphatidylserine are in a pharmaceutically acceptable carrier.

5. The treatment composition of claim 3, wherein said composition comprises at least one additional active ingredient selected from the group consisting of tyrosine, 1-dopa, Mucuna pruriens, phenylalanine, stimulants, and anxiolytics.

6. The treatment composition of claim 3 comprising taurine.

7. A treatment composition of claim 6 comprising glycine.

8. A treatment composition comprising an effective amount of Rhodiola Rosea and an effective amount of phosphorylated serine.

9. A method of treating a patient with over-stimulation due to the effects of stimulants, the method comprising administering an effective amount of Rhodiola Rosea and an effective amount of phosphatidylserine to the patient.

10. A method of treating a patient with over-stimulation resistant to a medication that prevents overstimulation, the method comprising administering an effective amount of Rhodiola Rosea and an effective amount of phosphatidylserine to the patient along with the medication.

11. The method of claim 1 wherein said patient has a disorder selected from the group consisting of insomnia, ADHD, obsessive/compulsive disorder, depersonalization disorder, post-traumatic stress disorder, panic disorder, opositional/defiance disorder, or internalizing or externalizing disorder.
